(19) United States
(12) Patent Application Publication
(10) Pub. No.: US 2023/0302200 A1
(43) Pub. Date: Sep. 28, 2023

(54) Autologous, prevascularized breast tissue constructs produced in a 3D printing method, and methods for producing same

(71) Applicant: Universitätsmedizin der Johannes Gutenberg-Universität Mainz, Mainz (DE)

(72) Inventors: Martin Heller, Mainz (DE); Walburgis Brenner, Mainz (DE)

(21) Appl. No.: 18/041,244

(22) PCT Filed: Aug. 10, 2021
(86) PCT No.: PCT/EP2021/072240

§ 371 Date: Feb. 10, 2023

(30) Foreign Application Priority Data
Aug. 12, 2020 (DE) 10 2020 004 900.1

(51) Int. Cl. A61L 27/38; A61L 27/36; B33Y 10/00; B33Y 80/00; B33Y 70/00

(52) U.S. Cl. CPC A61L 27/3886; A61L 27/3804; A61L 27/3808; A61L 27/3691; A61L 27/3839; B33Y 10/00; B33Y 80/00; B33Y 70/00; A61L 2430/04

(57) ABSTRACT

Autologous prevascularized breast tissue constructs created via 3D printing and methods for 3D printing autologous prevascularized breast tissue constructs. The method comprises steps of: (i) providing a triculture consisting of adipose mesenchymal stem cells, fibroblasts, and endothelial progenitor cells, (ii) mixing the triculture cells with a bioink composed of biopolymers, (iii) printing three-dimensional structures of the breast tissue construct using the triculture-added bioink from step (ii), where the cells of the triculture are pretreated with growth media before printing so that the endothelial progenitor cells differentiate into endothelial cells and the adipose mesenchymal stem cells differentiate into adipocytes. After 3D printing, the development of vascular-like structures is induced.

### TECHNICAL FIELD

The present invention is in the field of artificial breast tissue construction and relates to a method for three-dimensional (3D) printing of autologous prevascularized breast tissue constructs, and to a breast tissue construct obtainable by such a method.

### BACKGROUND OF THE INVENTION

Breast cancer is the most common cancer in women. Medical options are diverse and depend on the type, size, stage and histological findings of the cancer. Especially in early stages, an adjuvant approach in the form of surgical removal or radiation is the procedure of choice for treatment. Surgical removal of the malignancy in the breast is accomplished either by mastectomy or by removal of portions of the breast tissue in sano (Rutter, C. E, Park, H. S., Killelea, B. K & Evans, S. B. Growing Use of Mastectomy for Ductal Carcinoma-In Situ of the Breast Among Young Women in the United States, Ann. Surg. Oncol. 22, 2378-2386 (2015)). Mastectomy is usually followed by breast tissue reconstruction either immediately after tumor resection or at a later time (Panchal, H. & Matros, E. Current Trends in Postmastectomy Breast Reconstruction. Plast. Reconstr. Surg. 140, 7S-13S (2017); Jeevan, R. et al. Findings of a national comparative audit of mastectomy and breast reconstruction surgery in England. J. Plast. Reconstr. Aesthetic Surg. 67, 1333-1344 (2014)). If the patient decides to undergo breast reconstruction, two different procedures can be used: First, in autologous reconstruction, tissue can be harvested from body sites, such as the abdomen, back, or buttocks, which then replaces the breast tissue. In this case, the surrounding muscles and vessels are implanted as well (e.g., the latissimus dorsi flap, myocutaneous flaps) or flap surgery can be performed free (Dayan, J. H. & Allen, R. J. Lower Extremity Free Flaps for Breast Reconstruction. Plast. Reconstr. Surg. 140, 77S-86S (2017)). Alternatively, breast tissue can be replaced with heterologous implants. The latter consist either entirely of silicone, or of a silicone shell that can be filled with saline, for example, to provide gradual tissue expansion (Yoshida, S. H., Chang, C. C. Teuber, S. S. & Gershwin, M. E. Silicone and Silicone: Theoretical and Clinical Implications of Breast Implants, Regul. Toxicol. Pharmacol. 17, 3-18 (1993)). Most reconstructions are performed through the use of implants (Albornoz, C. R. et al. A paradigm shift in U.S. Breast reconstruction: increasing implant rates. Plast. Reconstr. Surg. 131, 15-23 (2013)) because, among other reasons, it is not possible to harvest the required tissue mass in all patients. Furthermore, this technique implies the creation of an additional wound and the associated pain and trauma for the patient, which is not the case with reconstruction by implants. In addition, flap surgery is not performed with equal frequency in all hospitals, making location usually a determining factor in the choice of treatment (Alderman, A. K. et al. Patterns and correlates of postmastectomy breast reconstruction by U.S. plastic surgeons: results from a national survey. Plast. Reconstr. Surg. 127, 1796-803 (2011); Schreuder, K. et al. Hospital organizational factors affect the use of immediate breast reconstruction after mastectomy for breast cancer in the Netherlands, The Breast 34, 96-102 (2017)).

Due to the challenges described above, various approaches have been sought in the past to artificially produce autologous tissue for the reconstruction of lost breast tissue. Tissue engineering is a promising approach in this context. With the help of this procedure e.g., autologous and functional tissues can be generated in vitro from very small tissue samples on the basis of specific carrier matrices such as collagen membranes. A special discipline of tissue engineering is 3D bioprinting, which makes it possible to print cells directly in biologically compatible bioinks in three-dimensional constructs and thus produce functional tissues.

Branching blood arteries and capillaries contribute to the complexity of 3D-printed organs, which is a particular challenge for 3D bioprinting. To fabricate an artificial organ or muscle section, the structure must be enriched with tissue-specific cells (Zhu W, Qu X, Zhu J, et al. Direct 3D bioprinting of prevascularized tissue constructs with complex microarchitecture. Biomaterials. 2017; 124:106-115; Jia W. Gungor-Ozkerim P S, Zhang Y S, et al. Direct 3D bioprinting of perfusable vascular constructs using a blend bionic. Biomaterials. 2016: 106:58-68), Using hydrogels or other biocompatible materials (bioinks) and cells, it is possible to produce accurate 3D models of organs. In 3D bioprinting, a main distinction is made between scaffold-based printing and scaffold-free printing (Badhshinejad A. D'Souza R M. A brief comparison between available bio-printing methods. In 2015 IEEE Great Lakes biomedical conference (GLBC) 1-3 (IEEE, 2015). 2015). Various approaches to scaffold-based bio-printing have been developed, for example, by first fabricating a 3D scaffold from biomaterials and then printing the cells into the structure. In addition, methods are also available in which the scaffold structure and the cells are printed simultaneously. In scaffold-free printing, the bioink includes different cells or tissue spheroids to directly punt them together with the bioink (Ong C S, Fukunishi T. Zhang H, et al. Biomaterial-free three-dimensional bioprinting of cardiac tissue using human induced pluripotent stem cell derived cardiomyocytes. Sci Rep. 2017; 7 (1):4566).

For printing vascular structures, two main approaches are followed in 3D printing, one is direct printing of vessels, the other is indirect printing of scaffold materials (e.g. gelatin, collagen) mixed in a hydrogel (Delta P, Ayan B. Ozbolat I T. Bioprinting for vascular and vascularized tissue biofabrication, Acta Biomater. 2017, 51:1-20). When the hydrogel scaffold structure solidifies, a hollow vascular structure remains on which endothelial cells are later cultured, This process is also called microextrusion and has the advantage that stable vascular structures and many different scaffold structures can be combined. Native materials such as fibrin or collagen are often used to print vascular structures (Hinton T J. Jallerat Q. Palchesko R N, et al. Three-dimensional printing of complex biological structures by freeform reversible embedding of suspended hydrogels. Sci Adv. 2015; 1 (9):el500758). in addition, there are also PEG-fibrinogen-based scaffold structures for the development of three-dimensional cardiac tissue constructs composed of pluripotent cell-derived cardiomyocytes (Maiullari F. Costantini M, Milan M. et al. A multi-cellular 3D bio-printing approach for vascularized heart tissue engineering based on HUVECs and iPSC-derived cardiomyocytes. Sci Rep. 2018; 8 (1):1-15). In breast cancer, several approaches and some methods for 3D printing based on breast tissue models are already known (Cieversey Chantell et al, 3D Printing Breast Tissue Models: A Review of Past Work and Directions for Future Work; Micromachines 2019, 10, 501).

WO 2015/152954 A1 describes a method for producing artificial tissue, including a 3D artificial breast tissue, to provide models for cancer therapy. For this purpose, the method uses an extrusion material-containing bioink comprising connective tissue cells and another extrusion material-containing bioink, the cancer cells. After an incubation period in a cell culture, the extrusion material is removed, allowing the cells to form a three-dimensional biological tumor model.

For the printing of prevascularized structures, different bioinks can be used, for example cell-free bioink or cell-containing bioink, which can be used for inkjet bioprinting, extrusion bioprinting or pulsed laser printing. For extrusion bioprinters, alginate-containing bioink has proven to be beneficial (van Duinen V, Trietsch S. J. Joore J, Vulto P. Hankemeier T. Microfluidic 3D cell culture: from tools to tissue models. Curr Opin Biotechnol 2015; 35:118-126). Cell-adhesive GelMA bioink is often used to disperse fibroblasts to form channel structures. In turn, the pure cell-containing bioinks enable a low viscosity printing solution, making them applicable in a wide range of bioprinting systems. However, such bioinks require sufficient biological signals (e.g., cell-adhesive matrix, carrier cell types) to stabilize cell-cell interactions after printing. WO 2019/122351 A1 describes bioinks based on nanocellulose or a polysaccharide hydrogel and a human tissue-specific extracellular matrix (ECM) material, where 3D printing is performed under physiological conditions.

The creation of a three-dimensional reconstruction of the breast, especially after a breast carcinoma resection, therefore represents a particular challenge. Nevertheless, the advantages outweigh the classical methods, especially plastic reconstruction, which is usually used after surgical resection of tumor tissue from the breast or complete breast removal. In addition to the physical and psychological stress for patients, aesthetically pleasing results also play a decisive role.

### DESCRIPTION OF THE INVENTION

Against this background, it is therefore the object of the present invention to provide a breast tissue construct and a method for its manufacturing in which reconstruction of the breast after tumor resection is possible without creating secondary trauma. This object is solved by a method with the features of claim 1. Preferred embodiments can be found in the subclaims.

The method according to the invention is based on complex autologous breast tissue constructs generated via 3D bioprinting. According to the process, autologous cells, i.e. cells taken from the patient, are used, whereby rejection reactions can be reduced to a minimum. The cells used according to the invention enable an organ-typical microenvironment, which ultimately ensures rapid vessel formation in the artificial breast tissue construct. The breast (fat) tissue constructs are produced using a complex triculture of primary mesenchymal stem cells or (pre-)adipocytes, fibroblasts and endothelial progenitor cells isolated from minute tissue samples or blood from the patient. Secondary trauma and associated comorbidities associated with autologous tissue graft harvesting are minimized.

A culture of pretreated adipose mesenchymal stem cells, fibroblasts and endothelial progenitor cells is mixed with a bioink consisting of biopolymers and printed to complex prevascularized breast structures. Either printing of vessel-like three-dimensional structures is performed, or capillary formation is induced by culturing after printing. According to the invention, the cells of the triculture are pretreated with growth medium prior to printing so that the endothelial progenitor cells differentiate into endothelial cells and the adipose mesenchymal stem cells differentiate into adipocytes. The vascular structures of the breast tissue construct are preferably printed with vasculogenic cells, preferably endothelial cells.

Thus, the method of 3D printing autologous prevascularized breast tissue constructs according to the invention comprises the following steps:
(i) Providing a triculture consisting of adipose mesenchymal stem cells, fibroblasts, and endothelial progenitor cells,
(ii) Mixing the triculture cells with a bioink composed of biopolymers,
(iii) Printing three-dimensional structures of the breast tissue construct using the triculture-added bioink from step (ii),
wherein the cells of the triculture are pretreated with growth media prior to printing so that the endothelial progenitor cells differentiate into endothelial cells and the adipose mesenchymal stem cells differentiate into adipocytes, and wherein the development of vessel-like structures is induced after 3D printing.

Preferably, the bioink consists of biopolymers, such as cellulose, alginate, mannitol, gelatin methacrylate and/or collagen I. The inventors were able to show that cultivation and printing of the various cell types of triculture in collagen-based bioinks is possible, which is a prerequisite for the formation of capillary-like three-dimensional structures of breast tissue. An additional option is the use of self-extracted extracellular matrix from adipose tissue (adECM adipose derived extracellular matrix), which—like the cells—can be autologously derived and used, for example, in a composition or mixture with other inks mentioned. The cells used in the invention showed high viability using adECM. Thus, it was demonstrated for the first time that breast (fat) tissue construction via a cell culture pre-treated with growth medium, consisting of adipose mesenchymal stem cells, fibroblasts and endothelial progenitor cells, is possible to print autologous breast tissue constructs. The method enables the production of prevascularized breast fat tissue constructs, i.e., tissue constructs that already have capillary-like structures composed of endothelial cells. For example, prevascularization can be incorporated into the tissue equivalent using two methods. First, primary endothelial cells along with fibroblasts and mesenchymal stem cells in the bioink composed of biopolymers are printed directly into the construct using 3D bioprinting, resulting in the formation of a finely branched network of capillary-like structures. Second, larger vessels are printed into the tissue construct in the form of a canal or tube system. This is done with the vessel-forming endothelial cells. Starting from these punted vascular channels, a finely branched vascular network can subsequently be created, which ultimately spans the entire tissue construct. By combining the two methods, tissue constructs are created that are interspersed with large and small vascular networks. Prevascularization is essential for sufficient and timely connection to the recipients vascular system after transplantation to ensure successful adherence or ingrowth and to provide adequate oxygen and nutrients to all areas of the tissue constructs or the cells contained therein.

The individual components of the bioink can vary in composition and concentration. Preferably, the bioink according to the invention contains collagen I so that the formation of vascular structures can be induced and the vitality of the cells can be improved. Preferably, a co-culture of several days, preferably 7 days, is performed in a collagen I-containing bioink to form vascular-like structures. The viability of a co-culture of endothelial cells and fibroblasts in a collagen I-containing compared to a collagen I-free bioink can be done, for example, using an MTT or Alamarblue assay. These assay systems are used to determine the metabolic activity of cells, which correlates with cell viability under certain conditions. The detection of cell viability is based on a reduction of the yellowish, water-soluble dye 3-(4,5-dimethyltiazol-2-yl)-2,5-diphenyltetrazolium bromide (MTT) into a blue-violet, water-insoluble formazan or, in the case of the Alamarblue assay, on a reduction of the blue resazurin to pink-colored resofurin.

The use of mesenchymal stem cells or progenitor cells in a complex breast fat tissue equivalent represents a central component of the present invention, since these are autologous cells isolated from minute tissue samples or blood from patients and made available for the procedure according to the invention. The collection here is so insignificant that secondary trauma or associated comorbidities, which occur when autologous tissue grafts are collected, are avoided entirely. Mesenchymal stem cells are present in adult tissues including bone marrow and adipose tissue. Stem cells are detectable by, their surface markers CD105, CD73, and CD90 in the absence of expression of CD34, CD45, CD14 or CD11b, CD19, and CD79a or HLA DR. They have the ability to differentiate into adipocytes, but also into osteoblasts or chondroblasts.

The autologous endothelial progenitor cells used in the triculture are preferably obtained from the patient's blood. The autologous fibroblasts are preferably obtained from a small oral mucosa biopsy of the patient. The autologous mesenchymal stem cells are preferably derived from the patient's adipose tissue. The triculture cells obtained in this way are first isolated prior to printing, expanded separately and stimulated to differentiate using appropriate growth factor-containing media.

In a preferred embodiment, already differentiated endothelial cells are used for prevascularization of tissue constructs in an independent preliminary experiment, microvascular endothelial cells isolated from the foreskin of young male patients were also used within the scope of the invention. However, this severely limits the field of application of the breast fat tissue constructs and makes them unsuitable for patients undergoing breast surgery, making the endothelial progenitor cells used in accordance with the invention particularly advantageous for 3D printing the prevascularized tissue construct. Similar to differentiated endothelial cells, endothelial progenitor cells are capable of forming complex vascular structures. A heterogeneous circulating cell population of endothelial progenitor cells, preferentially consisting of “iate” endothelial progenitor cells (late EPCs), is used for prevascularization of breast (fat) tissue constructs. The much larger subpopulaton of early EPCs, in turn, probably promotes endothelial repair processes, mediated by paracrine effects. For prevascularization of breast tissue, these cells play rather a minor role.

Endothelial progenitor cells derived from a patents blood are first cultured and then transferred to a gelatin-coated culture surface and a specific selection medium. The selection medium is preferably an endothelial cell growth medium, such as Lonza's EGM™ bullet kit. The adipose mesenchymal stem cells are stimulated to differentiate into adipocytes using, for example, AdipoMAX (Sigma-Aldrich) and selected by anti-CD34-coupled magnetic beads. Fibroblasts are isolated using known methodology, shown using artificial pre-vascularized mucosa equivalents (Heller et al. Tissue engineered pre-vascularized buccal mucosa equivalents utilizing a primary triculture of epithelial cells, endothelial cells and fibroblasts. Biomaterials 77: 2017-15 (2016)).

The differentiation and vitality of the endothelial cells is an essential factor for a promising performance of the process according to the invention and the resulting product. Therefore, in a preferred embodiment, it is envisaged that the endothelial cells are not printed directly as a suspension, but are printed in advance as spheroids or on microcarriers (e.g. on gelatin coated microcarriers). Cultivation on microcarriers increases the vitality and differentiation of the cells. As a result, the cells acquire a three-dimensional culture structure even before 3D bioprinting, which supports vessel formation after printing.

The invention further relates to an autologous prevascularized breast tissue construct generated via a 3D printing process comprising a three-dimensional structure of several different cell types consisting of endothelial cells differentiated from endothelial progenitor cells, adipocytes differentiated from adipose mesenchymal stem cells, and fibroblasts, wherein the breast tissue construct is obtainable by a process as described above.

The invention offers the possibility of producing autologous breast (fat) tissue without creating the secondary surgical trauma that is otherwise common. The collection of minute tissue samples or blood for isolation of the autologous triculture cells used in the method of the invention minimizes additional stress to patients Furthermore, tissue constructs of any size can be manufactured, allowing reconstruction of large tissue defects, such as those resulting from total breast removal. After the manufacturing process of the tissue constructs, they are sterile and can be used directly. In the process, different sizes and shapes can be produced through the various selectable hardware parameters of a 3D bioprint.

### BRIEF DESCRIPTION OF THE DRAWINGS

Further features and advantages of the present invention will become apparent from the following description of preferred embodiments with reference to the accompanying figures, in which:

FIG. 1 shows the formation of vessel-like structures after 7 days of culture in a collagen I-containing bioink,

FIG. 2 shows the viability of an endothelial cell-fibroblast co-culture in a collagen I-containing bioink compared with a collagen I-free bioink,

FIG. 3 shows the cultivation of endothelial cells on microcarriers to increase cell viability and differentiation.

### DETAILED DESCRIPTION OF PREFERRED EMBODIMENTS

The following embodiments serve to illustrate the invention. By no means is the invention limited to these embodiments. The invention also encompasses combinations of individual embodiments or any combinations of features of individual embodiments.

In the following embodiments, two different approaches are taken for the manufacturing of the prevascularized tissue constructs, which are based on either 1-channel, or 2-channel printing.

For the manufacturing of a finely branched network of capillary-like structures within smaller tissue constructs, for the first approach (1-channel) the detached cells are transferred together in equal parts into cell medium so that a triculture with a total cell concentration of 10⁶ cells/ml is achieved. The cell suspension is then mixed with the collagen-based bioink at a ratio of 1:10 using a Luer-lock syringe and Luer-lock adapter and transferred to a print cartridge. After inserting the cartridge: the 3D constructs are printed into a sterile well plate in a channel using a pressure of 9-15 kPa and a 25 G tip.

For the second approach, larger vessels in the form of a channel or tube system are printed into the tissue construct (1-channel and/or 2-channel). For this purpose, the cells are first separated after detachment from the culture vessels. In this process, mesenchymal stem cells or adipocytes and fibroblasts (biculture) are transferred together into a cell suspension and endothelial cells (monoculture) are transferred into another suspension, each with a total cell concentration of 10⁶ cells/ml. Subsequently, the different cell suspensions (monoculture and biculture) are mixed with the collagen-based bioink at a ratio of 1:10 as described above and divided into two print cartridges. For the procedure described here, 25 G pressure tips are also used. For 1-channel printing, a basic scaffold from the biculture is first printed to produce a connective tissue structure that has tubular recesses or porous structures, Subsequently, the endothelial cell-bioinks mixture is then used to print the vascular structures. In the 2-channel system, the basic scaffold is printed from the biculture and, simultaneously, using the second channel, vascular structures are printed with the endothelial cell monoculture. The results are summarized in the figures below.

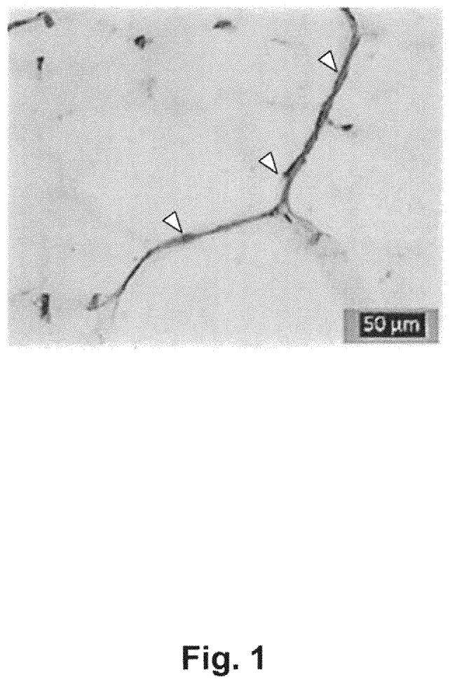

FIG. 1 depicts the morphology of endothelial cells on a collagen I-based bioink visualized by CD31 staining (here black). Vessel-like structures (white arrows) are shown, which form after 7-days co-culture of endothelial cells with fibroblasts in a collagen I-containing bioink.

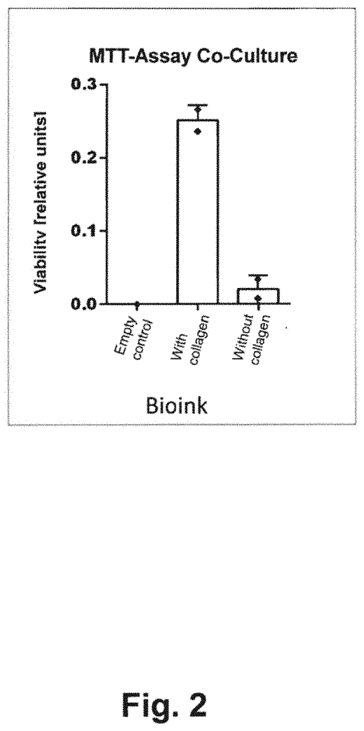

FIG. 2 shows the viability of an endothelial cell fibroblast co-culture in a collagen I-containing compared to a collagen I-free bioink, detected in an MTT assay. The collagen I content in the bioink is crucial for high viability.

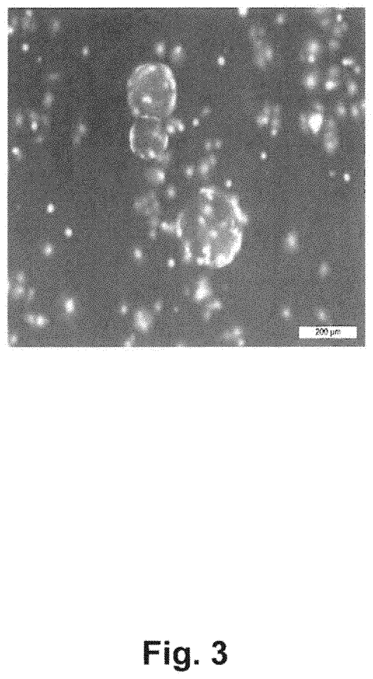

FIG. 3 shows endothelial cells cultured on gelatin-coated microcarriers, recognizable as bright staining (CD31). Adherence to microcarriers allows endothelial cells to retain high cell and differentiation.

1. A method for 3D printing autologous prevascularized breast tissue constructs comprising the steps of:
(i) Providing a triculture consisting of adipose mesenchymal stem cells, fibroblasts, and endothelial progenitor cells,
(ii) Mixing the triculture cells with a bioink composed of biopolymers,
(iii) Printing three-dimensional structures of the breast tissue construct using the triculture-added bioink from step (ii),
wherein the cells of the triculture are pretreated with growth media prior to printing so that the endothelial progenitor cells differentiate into endothelial cells and the adipose mesenchymal stem cells differentiate into adipocytes, and wherein the development of vessel-like structures is induced after 3D printing.

2. The method according to claim 1, characterized in that the development of vessel-like structures after 3D printing is induced with collagen I.

3. The method according to claim 1, characterized in that the 3D printing is performed in a 1-channel system and/or a 2-channel system.

4. The method according to claim 1, characterized in that the vascular structures of the breast tissue construct are printed with vessel-forming cells.

5. The method according to claim 15, characterized in that the endothelial cells are differentiated endothelial cells or microvascular endothelial cells.

6. The method according to claim 1, characterized in that the bioink comprises cellulose, alginate, mannitol, gelatin methacrylate and/or collagen I.

7. The method according to claim 1, characterized in that the primary mesenchymal stem cells, fibroblasts and/or endothelial progenitor cells are derived from autologous cells of a patient.

8. The method according to claim 7, characterized in that the endothelial progenitor cells are late endothelial progenitor cells from the patient's blood.

9. The method according to claim 7, characterized in that the endothelial progenitor cells are obtainable by culturing the cells taken from the blood for several days and converting them to a gelatin-coated culture surface with a selection medium.

10. The method according to claim 1, characterized in that the bioink is contained in a composition comprising self-extracted extracellular matrix from adipose tissue (adECM).

11. The method of claim 7, characterized in that the adipose mesenchymal stem cells are selected from a present adipose tissue sample of the patient by anti-CD34 coupled magnetic beads.

12. The method according to claim 7, characterized in that the fibroblasts are selected from a present oral mucosa sample of the patient.

13. The method according to claim 1, characterized in that the endothelial cells are cultured as spheroids or on microcarriers prior to printing.

14. An autologous prevascularized breast tissue construct generated via a 3D printing process, comprising a three-dimensional structure of several different cell types, consisting of
endothelial cells differentiated from endothelial progenitor cells,
adipocytes differentiated from adipose-derived mesenchymal stem cells and
fibroblasts,
wherein the breast tissue construct is obtainable by a method according to claim 1.

15. The method according to claim 4, characterized in that the vessel-forming cells are endothelial cells.
